(19) United States
(12) Patent Application Publication
(10) Pub. No.: US 2015/0210963 A1
(43) Pub. Date: Jul. 30, 2015

(54) Systems and Methods for Treating a Surface

(71) Applicant: The Procter & Gamble Company, Cincinnati, OH (US)

(72) Inventors: Alan David Willey, Cincinnati, OH (US); Jacob Robert Adams, Cincinnati, OH (US); Kady Lynn Willison, Cincinnati, OH (US); Kenneth Edward Yelm, Hamilton, OH (US); Gregory Mark Bunke, Lawrenceburg, IN (US); Robb Richard Gardner, Cleves, OH (US)

(21) Appl. No.: 14/594,187

(22) Filed: Jan. 12, 2015

Related U.S. Application Data
(60) Provisional application No. 61/931,008, filed on Jan. 24, 2014.

(51) Int. Cl. C11D 3/395; B08B 3/10; A61L 2/08; C11D 3/04; A01N 59/00

(52) U.S. Cl. CPC C11D 3/3951; C11D 3/046; C11D 3/3953; A01N 59/00; A61L 2/088; B08B 3/10

(57) ABSTRACT

Systems for treating a surface comprise a first composition and a second composition. The first composition comprises a water soluble organic photoactivator. The second composition comprises an electron acceptor which accepts an electron from the photoactivator when the photoactivator is in a photo-excited state and/or reduced state and a benefit active precursor which converts into a benefit active agent via electron transfer. Methods for treating a surface comprise applying the first composition to the surface, applying the second composition to the surface, and exposing the surface to light.

### FIELD OF THE INVENTION

The present invention relates to systems for treating a surface that include one or more photoactivators to generate one or more benefit active agents, effective as a bleaching agent, stain remover, or antimicrobial and/or in eliminating biofilm. The present invention also relates to methods for cleaning and/or bleaching surfaces, and for providing a method of disinfecting or sanitizing surfaces and/or removing biofilm.

### BACKGROUND OF THE INVENTION

Cleaning compositions are used throughout the world in people's homes and workplaces. These compositions range from surface cleaners and disinfectants to bleach for removing stains from one's clothes or teeth. However, conventional cleaning and whitening compositions are limited by the standard chemistry which generates the cleaning or whitening attribute of the composition.

Conventional low cost cleaners, such as chlorine bleach (sodium hypochlorite), are limited in their ability to disinfect and sanitize. For example, such systems have limited benefit on biofilms, a complex biological community formed extensively in the natural environment by bacteria.

Another attempt at eliminating biofilm is through the production of chlorine dioxide and other biocidal gases. Specifically, it is known that chlorine dioxide can be generated by mixing a chlorine dioxide precursor, such as a metal chlorite, and an activator component, such as a transition metal or acid. When each of the components are combined the chlorine dioxide precursor and activator component react to form chlorine dioxide. Such reactions are highly volatile and toxic and are, therefore, not desirable for home applications. Furthermore, these components must be sequestered to prevent premature formation of the chlorine dioxide. However, multi-compartment packaging is more expensive and can still allow premature mixing of the components and accidental generation of chlorine dioxide. As such, such systems are undesirable.

Yet another attempt at eliminating biofilm is through the use of a photoactivator to produce chlorine dioxide. Specifically, it is known to use titanium dioxide (TiO₂) and a chlorine dioxide precursor in conjunction with exposure to ultraviolet light to generate chlorine dioxide. However, such processes are undesirable due to the health risks associated with exposure to ultraviolet light, the degradation which can occur to the other components of the cleaning compositions, and the use of an insoluble inorganic photoactivator. In addition, titanium dioxide forms particulates which leave undesirable residue on surfaces and requires additives to suspend in and imparts opaqueness to compositions.

As such, there remains a need for a system that includes a water-soluble photoactivator that can enable the generation of one or more benefit active agents effective as a bleaching agent, stain remover, or antimicrobial and/or in eliminating biofilm. There further remains a need for a system that includes a water-soluble photoactivator that produces a substantially colorless composition that is effective as a bleaching agent, stain remover, or antimicrobial and/or in eliminating biofilm and activatable by visible light.

### SUMMARY OF THE INVENTION

The present invention, in one aspect, relates to a system for treating a surface, the system comprising a first composition and a second composition. The first composition comprises a water soluble organic photoactivator and the second composition comprises an electron acceptor which accepts an electron from the photoactivator when the photoactivator is in a photo-excited state and/or reduced state and a benefit active precursor which converts into a benefit active agent via electron transfer.

In another aspect, the present invention relates to a method for treating a surface comprising applying a first composition to the surface, applying the second composition to the surface, and exposing the surface to light.

The present invention further relates to methods of cleaning surfaces, bleaching stains, disinfecting surfaces, and removing biofilms.

It has now been surprisingly found that providing a system according to the present invention enables the generation of one or more benefit active agents effective as a bleaching agent, stain remover, or antimicrobial and/or in eliminating biofilm. It has also now been surprisingly found that providing a system of the present invention, can produce a composition that is effective as a bleaching agent, stain remover, or antimicrobial and/or in eliminating biofilm.

### BRIEF DESCRIPTION OF THE DRAWINGS

FIG. 1 is a schematic representing reactions involving the compositions and methods of the present invention.

### DETAILED DESCRIPTION OF THE INVENTION

The present invention relates to systems that include water soluble photoactivators. Furthermore, the present invention also relates to system comprising a first composition comprising a photoactivator, and a second composition comprising an electron acceptor and a benefit active precursor. Still further, the present invention also relates to methods for cleaning and/or bleaching surfaces, and for providing a method of disinfecting or sanitizing surfaces and/or eliminating biofilm using a photoactivator, an electron acceptor and a benefit active precursor.

### First Composition

The first composition of the system of the present invention comprises a water soluble photoactivator, as described herein.

### Photoactivator

The water soluble photoactivators of the present invention may comprise a photoactive moiety and a hydrophilic moiety. For purposes of the present invention, the term “hydrophilic moiety” refers to a moiety that is attracted to water and dissolves in water to form a homogenous solution. In one embodiment, the hydrophilic moiety is selected from the group consisting of water soluble oligimers, water soluble polymers and water soluble copolymers. In one preferred embodiment, the hydrophilic moiety may be selected from the group consisting of alkylene oxide oligimers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, polysaccharides, 2-ethyl-2-oxazoline, hydroxyethyl methacrylate, vinyl pyridine-N-oxide, diallyl dimethyl ammonium chloride, maleic acid, lysine, arginine, histidine, aspartic acid, glutamic acid, serine, threonine, asparagine, glutamine, isopropyl acrylamide, styrene sulfonic acid, vinyl methyl ether, vinyl phosphoinic acid, ethylene imine, and mixtures thereof. In one especially preferred embodiment, the hydrophilic moiety may be selected from the group consisting of alkylene oxide oligimer polymers, alkylene oxide oligimer copolymers, vinyl alcohol, vinyl pyrrolidone, acrylic acid, acrylamide, cellulose, and mixtures thereof. For purposes of the present invention, the term “photoactive moiety” refers to an organic conjugated moiety that is capable of absorbing a photon of light and thereby forming an excited state (singlet or triplet). It will be understood that the term “photoactive moiety” does not, however, refer to a charge-transfer excited state. It will further be understood that the photoactive moieties, as disclosed herein, may include a single moiety or a combination of two, three, four or any other number of moieties, as known in the art.

In one embodiment of the present invention, the photoactive moiety is selected from the group consisting of 1,1′-biphenyl-4,4′-diamine, 1,1′-biphenyl-4-amine, benzophenone, 1,1′-biphenyl-4,4′-diol, 1,1′-biphenyl-4-amine, 1,1′-biphenyl-4-ol, 1,1′:2′,1″-terphenyl, 1,1′:3′,1″-terphenyl, 1,1′:4′,1″:4″,1′″-quaterphenyl, 1,1′:4′,1″-terphenyl, 1,10-phenanthroline, 1,1′-biphenyl, 1,2,3,4-dibenzanthracene, 1,2-benzenedicarbonitrile, 1,3-isobenzofurandione, 1,4-naphthoquinone, 1,5-naphthalenediol, 10H-phenothiazine, 10H-phenoxazine, 10-methylacridone, 1-acetonaphthone, 1-chloroanthraquinone, 1-hydroxyanthraquinone, 1-naphthalenecarbonitrile, 1-naphthalenecarboxaldehyde, 1-naphthalenesulfonic acid, 1-naphthalenol, 2(1H)-quinolinone, 2,2′-biquinoline, 2,3-naphthalenediol, 2,6-dichlorobenzaldehyde, 21H,23H-porphine, 2-aminoanthraquinone, 2-benzoylthiophene, 2-chlorobenzaldehyde, 2-chlorothioxanthone, 2-ethylanthraquinone, 2H-1-benzopyran-2-one, 2-methoxythioxanthone, 2-methyl-1,4-naphthoquinone, 2-methyl-9(10-methyl)-acridinone, 2-methylanthraquinone, 2-methylbenzophenone, 2-naphthalenamine, 2-naphthalenecarboxylic acid, 2-naphthalenol, 2-nitro-9(10-methyl)-acridinone, 9(10-ethyl)-acridinone, 3,6-qcridinediamine, 3,9-dibromoperylene, 3,9-dicyanophenanthrene, 3-benzoylcoumarin, 3-methoxy-9-cyanophenanthrene, 3-methoxythioxanthone, 3′-methylacetophenone, 4,4′-dichlorobenzophenone, 4,4′-dimethoxybenzophenone, 4-bromobenzophenone, 4-chlorobenzophenone, 4′-fluoroacetophenone, 4-methoxybenzophenone, 4′-methylacetophenone, 4-methylbenzaldehyde, 4-methylbenzophenone, 4-phenylbenzophenone, 6-methylchromanone, 7-(diethylamino)coumarin, 7H-benzldelanthracen-7-one, 7H-benzolclxanthen-7-one, 7H-furo[3,2-g][1]benzopyran-7-one, 9(10H)-acridinone, 9(10H)-anthracenone, 9(10-methyl)-acridinone, 9(10-phenyl)-acridinon, 9,10-anthracenedione, 9-acridinamine, 9-cyanophenanthrene, 9-fluorenone, 9H-carbazole, 9H-fluoren-2-amine, 9H-fluorene, 9H-thioxanthen-9-ol, 9H-thioxanthen-9-one, 9H-thioxanthene-2,9-diol, 9H-xanthen-9-one, acetophenone, acridene, acridine, acridone, anthracene, anthraquinone, anthrone, α-tetralone, benz[a]anthracene, benzaldehyde, benzamide, benzo[a]coronene, benzo[a]pyrene, benzo[f]quinoline, benzo[ghi]perylene, benzo[rst]pentaphene, benzophenone, benzoquinone, 2,3,5,6-tetramethyl, chrysene, coronene, dibenz[a,h]anthracene, dibenzo[b,def]chrysene, dibenzo[c,g]phenanthrene, dibenzo[def,mno]chrysene, dibenzo[def,p]chrysene, DL-tryptophan, fluoranthene, fluoren-9-one, fluorenone, isoquinoline, methoxycoumarin, methylacridone, michler's ketone, naphthacene, naphtho[1,2-g]chrysene, N-methylacridone, p-benzoquinone, p-benzoquinone, 2,3,5,6-tetrachloro, pentacene, phenanthrene, phenanthrenequinone, phenanthridine, phenanthro[3,4-c]phenanthrene, phenazine, phenothiazine, p-methoxyacetophenone, pyranthrene, pyrene, quinoline, quinoxaline, riboflavin 5′-(dihydrogen phosphate), thioxanthone, thymidine, xanthen-9-one, xanthone, derivatives thereof, and mixtures thereof.

Preferably, the photoactive moiety is selected from the group consisting of xanthone, xanthene, thioxanthone, thioxanthene, phenothiazine, fluorescein, benzophenone, alloxazine, isoalloxazine, flavin, derivatives thereof, and mixtures thereof. In one preferred embodiment, the photoactive moiety is thioxanthone.

Other suitable water-soluble photoactivators for the compositions of the present invention include fluoresceins and derivatives thereof; preferably halogen substituted fluoresceins; more preferably bromo- and iodo-fluoresceins such as dibromo fluorescein, diodo fluorescein, rose bengal, erythrosine, eosin (e.g. Eosin Y).

It is a further aspect of the present invention that the photoactivator preferably comprises less than about 35%, about 30%, about 25%, about 20%, about 15%, about 10%, about 5%, about 3% and about 2%, by weight of the photoactivator, of the photoactive moiety. As such, the photoactivator preferably comprises at least about 65%, about 70%, about 75%, about 80%, about 85%, about 90%, about 95%, about 97%, and about 98%, by weight of the photoactivator, of hydrophilic moiety. In one aspect, the photoactivator comprises less than about 2%, by weight of the photoactivator, of photoactive moiety (such as thioxanthone), and at least about 98%, by weight of the photoactivator, of hydrophilic moiety (such as polyethylene glycol). Without wishing to be bound by theory, it is believed that such a photoactivator not only is water soluble, but will resist aggregation due to the steric hindrance imparted by the hydrophilic moiety or any other non-photoactive moiety.

It is still further another aspect of the present invention that the photoactive moiety has an absorption band between about 350 nm and about 750 nm, about 350 nm and about 600 nm, about 350 nm and about 420 nm, and about 380 nm and about 400 nm.

In another embodiment, the photoactive moiety does not have an absorption band between about 420 nm and about 720 nm, about 500 and about 700 nm, about 500 nm and about 650 nm, and about 500 nm and about 600 nm. In this embodiment, it will be understood that the photoactivator will be substantially colorless to the human eye when used in an aqueous solution at a concentration of about 500 ppm.

In yet another aspect of the present invention, the photoactivator can be activated to a photo-excited state by excitation with incident radiation of a wavelength greater than 350 nm, preferably between about 350 nm and about 420 nm. In one embodiment, the photo-excited state lifetime is greater than about 0.5 nanosecond, 1 nanosecond, 10 nanoseconds, 50 nanoseconds, 100 nanoseconds, 300 nanoseconds and 500 nanoseconds. In another embodiment, the photo-excited state of the photoactivator has an energy greater than about 100 kJ/mol, 150 kJ/mol, 200 kJ/mol and 300 kJ/mol more than a ground state of the photoactivator.

In one embodiment, the photoactivator can be excited to a “singlet state” and in another a “triplet state”, as both of those terms are known in the art.

In yet another embodiment, the present invention relates to a photoactivator having the formula:

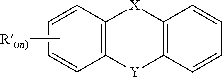

wherein,
X is selected from the group consisting of C, O, NH, C═O, CH₂, CHR″, CR″R′″, S, SO, and SO₂;
Y is selected from the group consisting of C, O, NH, C═O, CH₂, CHR″, CR″R′″, S, SO, and SO₂;
R′, R″ and R′″ may be —H or selected from a group of substituents that include a moiety selected from the group consisting of Oxygen, Nitrogen, Sulfur, Halogen and Hydrocarbon;
at least one of R′, R″ or R′″ further comprises a hydrophilic moiety R;
R is selected from the group consisting of water soluble oligimers, water soluble polymers and water soluble copolymers;
m is an integer from 0-8; and
the combined molecular weight of the substituents R′, R″ and R′″ is greater than 400 atomic mass units (AMU).

It can be appreciated by one of ordinary skill in the art that the substituent(s) R′ as depicted in the formula above reflects that the substitution of the photoactivator may include any number of substituents from zero to eight and that these substituents may be covalently attached to the peripheral carbon atoms of the photoactivator. Where m>1, the multiple R′ groups can be independently selected from a group of substituents that include a moiety selected from the group consisting of Oxygen, Nitrogen, Sulfur, Halogen and Hydrocarbon.

In one embodiment, R may be selected from the group consisting of alkylene oxide oligimers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, polysaccharides, 2-ethyl-2-oxazoline, hydroxyethyl methacrylate, vinyl pyridine-N-oxide, diallyl dimethyl ammonium chloride, maleic acid, lysine, arginine, histidine, aspartic acid, glutamic acid, serine, threonine, asparagine, glutamine, isopropyl acrylamide, styrene sulfonic acid, vinyl methyl ether, vinyl phosphoinic acid, ethylene imine, and mixtures thereof.

R′, R″ and R′″ moieties that may replace hydrogen and which contain only carbon and hydrogen atoms include any hydrocarbon moieties, as known in the art, including, alkyl, alkenyl, alkynyl, alkyldienyl, cycloalkyl, phenyl, alkyl phenyl, naphthyl, anthryl, phenanthryl, fluoryl, steroid groups, and combinations of these groups with each other and with polyvalent hydrocarbon groups such as alkylene, alkylidene and alkylidyne groups. Specific non-limiting examples of such groups are:

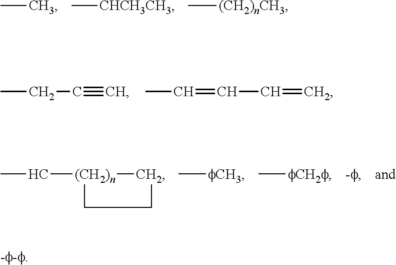

where n is independently chosen as being from 0-22

R′, R″ and R′″ moieties containing oxygen atoms that may replace hydrogen include hydroxy, acyl or keto, ether, epoxy, carboxy, and ester containing groups. Specific non-limiting examples of such oxygen containing groups are:

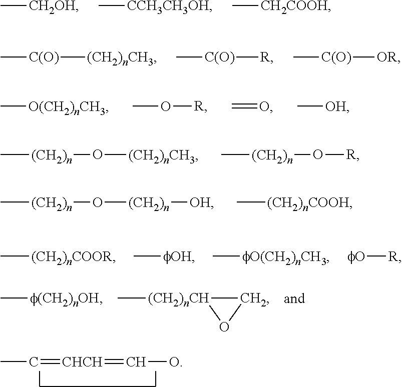

where n is independently chosen as being from 0-22

R′, R″ and R′″ moieties containing sulfur atoms that may replace hydrogen include the sulfur-containing acids and acid ester groups, thioether groups, mercapto groups and thioketo groups. Specific non-limiting examples of such sulfur containing groups are:

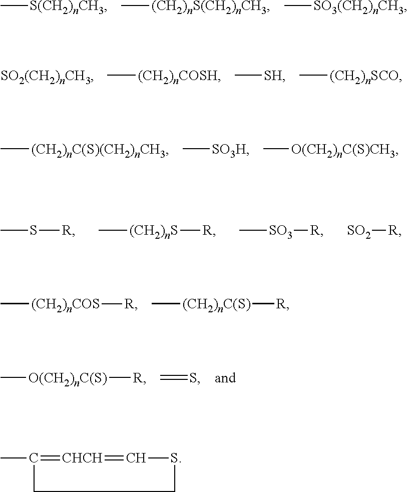

where n is independently chosen as being from 0-22

R′, R″ and R′″ moieties containing nitrogen atoms that may replace hydrogen include amino groups, the nitro group, azo groups, ammonium groups, amide groups, azido groups, isocyanate groups, cyano groups and nitrile groups. Specific non-limiting examples of such nitrogen containing groups are:
—NH₂, —NH₃⁺, —NH(CH₂)ₙCH₃, —N((CH₂)ₙCH₃)₂, —(CH₂)ₙNH(CH₂)ₙCH₃, —(CH₂)ₙN((CH₂)ₙCH₃)₂, —CH₂CONH₂, —CH₂CONH(CH₂)ₙCH₃, —CH₂CON((CH₂)ₙCH₃)₂, —NRH₂⁺, —NH—R, —NR₂, —(CH₂)ₙNH—R, —(CH₂)ₙNR₂, —(CH₂)ₙCONH—R, —(CH₂)ₙCONR₂, —(CH₂)ₙCON₃, —(CH₂)ₙCH═NOH, —CN, —CH(CH₂)ₙNCO, —(CH₂)ₙNCO, —Nφ, -φN═NφOH, and ≡N.

where n is independently chosen as being from 0-22.

R′, R″ and R′″ moieties containing halogen atoms that may replace hydrogen include chloro, bromo, fluoro, iodo groups and any of the moieties previously described where a hydrogen or a pendant alkyl group is substituted by a halo group to form a stable substituted moiety. Specific non-limiting examples of such halogen containing groups are: —Cl, —Br, —I, —(CH₂)ₙCOCl, -φF₅, -φCl, —CF₃, and —(CH₂)ₙφBr.

It is understood that any of the above moieties that may replace hydrogen can be substituted into each other in either a monovalent substitution or by loss of hydrogen in a polyvalent substitution to form another monovalent moiety that can replace hydrogen in the organic compound or radical.

As used herein “φ” represents a phenyl ring.

Other suitable photoactivators for use in the system of the present invention are described in detail in U.S. Application Ser. No. 61/930,999, filed Jan. 24, 2014, entitled “PHOTOACTIVATORS” (Attorney Docket No. 13058P).

### Second Composition

The second composition of the system of the present invention comprises an electron acceptor and a benefit active precursor, as described herein.

### Electron Acceptor

The photocatalyzable consumer product composition of the present invention comprises an electron acceptor. It will be understood to those skilled in the art that photocatalytic reduction and oxidation chemistries differ from conventional, energy-transfer photochemistry in that the photocatalytically-induced transfer of electrons can result in chemical transformation of reagents (e.g. transformation of the benefit precursor material to the benefit active) and oxidation of the benefit precursor material to produce a benefit active which is capable of providing a beneficial result, for example, cleaning, disinfection, bleaching, and/or whitening.

For the purposes of the present invention the term “electron acceptor” is defined as “a compound or moiety which accepts an electron from the photoactivator when the photoactivator is in a photo-excited state and/or one electron reduced state.” This electron transfer process is normally a very rapid and reversible process.

The ability of the electron acceptor to accept an electron from the excited photoactivator is generally described in Turro, N. J., V. Ramamurthy, and J. C. Scaiano, Principles of Molecular Photochemistry: An Introduction, Chapter 7, p. 41 (University Science Books 2009, Paperback edition). It is understood that the reaction between the reactants is favored when the Gibbs free energy (delta G) is less than 0.

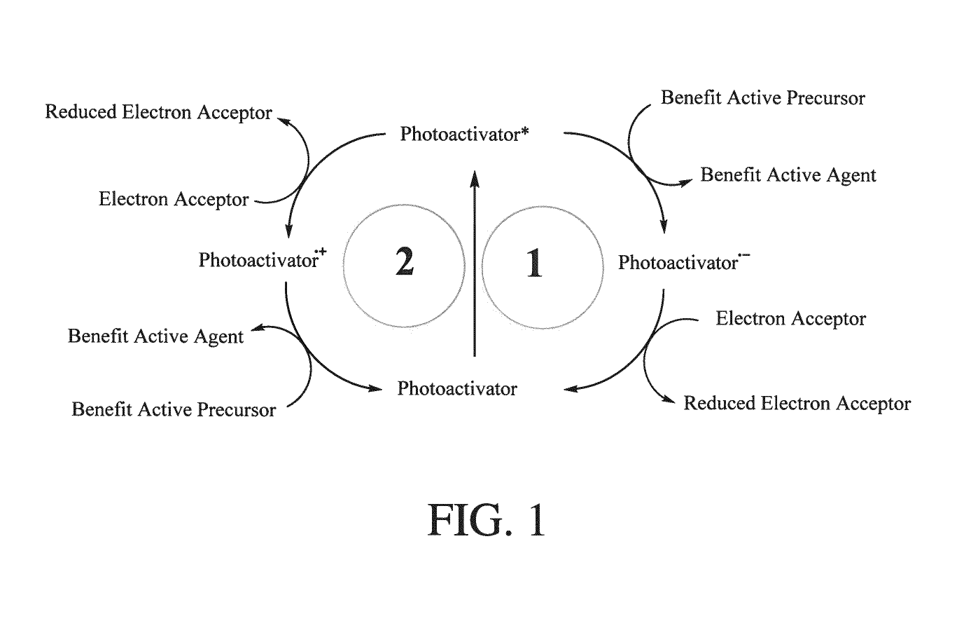

The reaction process is exemplified schematically in FIG. 1. As shown in FIG. 1, Reaction 1 (the right half of the figure) illustrates a reaction in which electron transfer occurs from a benefit active precursor to the excited state of the photoactivator (thereby forming a benefit active) and then from the one-electron reduced form of the photoactivator to the electron acceptor as described herein. As shown in FIG. 1, Reaction 2 (the left half of the figure) illustrates a reaction in which electron transfer occurs from the excited state of the photoactivator to the electron acceptor and then from the one electron oxidized form of the photoactivator to the benefit active precursor (thereby forming a benefit active). In all cases, the Gibbs free energy for the electron transfer should be less than 0. It is understood that the conversion of the photoactivator to its photoactivated state (“Photoactivator*”) is initiated by the absorption of light, which is also present in the reaction.

It will further be understood to those skilled in the art that any electron transfer between species comprising the photocatalyzable consumer product composition further requires effective Brownian collision to occur between the reacting species and that effective electron transfer between the photochemically excited state of the photoactivator and any species comprising the photocatalyzable consumer product composition (e.g. the electron acceptor) may further depend on the lifetime of the excited state of the photoactivator, the concentration of the photoactivator, and the concentration of the electron acceptor.

The electron acceptor of the present invention may be any species that accepts an electron from the photoactivator when the photoactivator is in a photo-excited state and/or reduced state. The electron acceptor must be present in the photocatalyzable consumer product composition in sufficient concentration to enable Brownian collisions with the photoactivator, given the concentration of the photoactivator and the lifetime of the photochemically excited state of the photoactivator.

A suitable electron acceptor can be selected from the group consisting of:

Viologens: e.g., methyl viologen;

Biyridiums: e.g., 2,2′ bipyridinium, 3,3′ bipyridinium, 3,4′ bipyridinium;

Quinones: e.g., para-Benzoquinone, 2,3-Dichloro-5,6-dicyano-p-benzoquinone, Tetrahydroxy-1,4-quinone hydrate, 2,5-di-tert-butylhydroquinone, tert-Butylhydroquinone, Anthraquinone, Diaminoanthroquinone, Anthraquinone-2-sulfonic acid;

Polycyclic aromatic hydrocarbons: e.g., Naphthalene, Anthracene, Pyrene, Dicyanobenzene, dicyano naphthalene, dicyano anthracene, dicyanopyrene;

Transition metal salts: e.g., Chloropentaamine cobalt dichloride, Silver nitrate, Iron Sulfate, copper sulfate;

Nanoparticle semiconductors: e.g., Titanium Dioxide, Zinc Oxide, Cadmium Selenide;

Persulfates: e.g., Ammonium persulfate, Sodium persulfate, Potassium persulfate;

Nitroxyl radicals: e.g., (2,2,6,6-Tetramethylpiperidin-1-yl)oxy, Dimethylthiourea, Tetranitromethane, Lithium, sodium and potassium acetoacetate, Oxaloacetic acid;

Ascorbic acid salts: e.g., Sodium ascorbate;

Phenols: 2,6-Dicholorophenolindophenol, 4-methoxyphenol;

Others: 4-Methylmorpholine N-oxide, 4-tert-Butylcatechol, Allopurinol, Pyridoxal 5′-phosphate, pyridoxal hydrochloride, Sodium benzoate, Sodium Nitrate, Sodium Nitrite, Diatomic Oxygen; and

Mixtures thereof.

With respect to suitable electron acceptors, diatomic oxygen is an electron acceptor which can be present in the composition due to dissolution of oxygen from the atmosphere into the composition, especially in an aqueous liquid composition. Most aqueous liquid compositions will have a sufficient content of diatomic oxygen as an electron acceptor to enable the electron transfer process. This can be enhanced with the addition of other electron acceptors in the composition as an ingredient. With respect to solid compositions (or other substantially anhydrous compositions), such compositions typically will not have a sufficient level of diatomic oxygen to enable the electron transfer process. Therefore, a solid composition which does not contain an electron acceptor as an added ingredient to the composition can nonetheless be photochemically active upon dissolution of the solid composition into an aqueous solution due to the presence of diatomic oxygen in the aqueous solution (e.g. a solid detergent composition that is dissolved in water can form an aqueous solution containing diatomic oxygen at a level sufficient to enable the electron transfer process). The present invention therefore encompasses a solid composition comprising a water soluble photoactivator and an oxyhalite, without an electron acceptor being added to the composition as an ingredient. Such a solid composition can be photoactivated upon dissolution in water wherein diatomic oxygen can serve as the electron acceptor.

With respect to suitable electron acceptors, nanoparticle semiconductors such as titanium dioxide can be used at relatively low levels to serve as electron acceptors, preferably less than about 1%, preferably less than 0.5%, preferably less than 0.1%, preferably less than 0.05%, preferably less than 0.01%, by weight of the consumer product composition. At higher levels, such materials may function efficiently as photoactivators, however any use of nanoparticle semiconductors in the present invention is preferably at a low enough level such that the material does not function efficiently as a photoactivator to provide significant consumer noticeable benefits and functions instead as an electron acceptor.

The photocatalyzable consumer product composition is preferably an aqueous composition and the electron acceptor is preferably a water soluble species selected from one or more of the groups listed above.

### Benefit Active Precursor

The photocatalyzable consumer product composition of the present invention comprises a benefit active precursor. When used in the photocatalyzable consumer product composition of the present invention and exposed to appropriate light (such as in the methods of the present invention), the benefit active precursor is converted into a benefit active (such as chlorine dioxide). The benefit active is the one electron oxidation product(s) of the benefit active precursor.

In one aspect of the present invention, the benefit active precursor is a material selected from one or more species according to the following formula:

A[XOₙ]ₘ

wherein
A is selected from the group consisting of monovalent cations, divalent cations, and trivalent cations; A can be an organic or inorganic cation; A is preferably selected from the group consisting of Aluminum, Barium, Calcium, Cobalt, Chromium, Copper, Iron, Lithium, Potassium, Rubidium, Magnesium, Manganese, Molybdenum, Nickel, Sodium, Titanium, Vanadium, Zinc, ammonium, alkyl-ammonium, aryl-ammonium, and mixtures thereof; A is more preferably selected from the group consisting of lithium, sodium, potassium, magnesium, calcium, ammonium, and mixtures thereof;
X is selected from the group consisting of chlorine, bromine, iodine, and mixtures thereof;
n is 1, 2, 3, or 4, preferably n is 2, 3, or 4; and
m is 1, 2, or 3.

The benefit active precursor of the present invention is preferably an oxyhalite, and is preferably selected from the group consisting of hypochlorite salts, chlorite salts, chlorate salts, perchlorate salts, hypobromite salts, bromite salts, bromate salts, perbromate salts, hypoiodate salts, iodite salts, iodate salts, periodate salts and mixtures thereof. Suitable benefit active precursors include those selected from the group consisting of sodium chlorite, sodium bromite, sodium iodite, potassium chlorite, potassium bromite, potassium iodite, sodium chlorate, sodium bromate, sodium iodate, potassium chlorate, potassium bromate, potassium iodate, sodium hypochlorite, sodium hypobromite, sodium hypoiodite, sodium perchlorate, potassium perchlorate, and mixtures thereof. In at least one aspect, the benefit active precursor is not a hypo-halite, such as hypochlorite.

In one aspect, the benefit active precursor may be a chlorite salt. A specific example of a chlorite salt suitable for use as a benefit active precursor is sodium chlorite (NaClO₂). In this embodiment, activation of the chlorite salt through transfer of an electron to the photoactivated photocatalyst results in the formation of the benefit active chlorine dioxide (ClO₂). Chlorine dioxide is a potent biocide and bleaching agent. In addition to salts, various other precursor forms are contemplated herein.

### Photocatalyzable Consumer Product Composition

The present invention also relates to photocatalyzable consumer product compositions that include the photoactivator, as described in further detail above, an electron acceptor and a benefit active precursor. As used herein, consumer product compositions encompass beauty care compositions, fabric and home care compositions, and health care compositions. Beauty care compositions generally include compositions for treating hair, including, bleaching, coloring, dyeing, conditioning, growing, removing, retarding growth, shampooing, styling; deodorants and antiperspirants; personal cleansing; color cosmetics; products, and/or methods relating to treating skin, including application of creams, lotions, and other topically applied products for consumer use; and products and/or methods relating to orally administered materials for enhancing the appearance of hair, skin, and/or nails; and shaving. Fabric and home care compositions generally include compositions for treating fabrics, hard surfaces and any other surfaces in the area of fabric and home care, such as car care, dishwashing, fabric conditioning (including softening), laundry detergency, laundry and rinse additive and/or care, hard surface cleaning and/or treatment, and other cleaning for consumer or institutional use. Oral care compositions generally include compositions for use with any soft and/or hard tissue of the oral cavity or conditions associated therewith, e.g., anti-caries compositions, anti-microbial compositions, anti-plaque chewing gum, compositions, breath compositions, confectionaries, dentifrices/toothpastes, denture compositions, lozenges, rinses, and tooth whitening compositions.

The photocatalyzable consumer product composition may be an aqueous solution, a solid, or incorporated into a material, such as a film. In another embodiment, the individual components of the photocatalyzable consumer product composition may be incorporated into both an aqueous solution and a material, such as a film. In one embodiment, the photoactivator may be included in a film and the electron acceptor and/or benefit active precursor maybe included in an aqueous solution. It will be understood that in this particular embodiment, a film comprising a photoactivator may be applied to surface and an aqueous solution comprising an electron acceptor and benefit active precursor may be applied separately.

However, if the photocatalyzable consumer product composition is an aqueous composition, the composition may comprise from 1% to 99%, by weight of the composition, of water. It will therefore be understood that the photocatalyzable consumer product composition can be in concentrated or diluted form. It is further contemplated that all or a portion of the water may be replaced with another solvent such as ethanol, glycol, glycol-ethers, glycerin, water soluble acetates and alcohols.

As noted above, the present invention relates to photocatalyzable consumer product compositions that include the photoactivator, an electron acceptor and a benefit active precursor. In such embodiments it will be understood that the photocatalyst can be excited into a singlet and/or triplet state via activation by light in the visible wavelengths. It will also be understood that the benefit active precursor can be converted into a benefit active agent upon triggering by the photocatalyst in an activated singlet and/or triplet state after exposure to visible light. It will be understood that the photocatalyst is unreactive with the benefit active precursor without activation by light.

The photocatalyzable consumer product composition is a system responsive to light; for example, visible, ultraviolet and/or infrared. In one preferred embodiment, the system is responsive visible light. In the present embodiment, photon transfer from the light source to the photocatalyst allows the reaction to progress to create an effective benefit agent that, in some embodiments, may act to clean, disinfect or sanitize, and/or bleach or whiten.

### Optional Additives

The photocatalyzable consumer product compositions of the invention may also contain additional adjunct additives. The precise nature of these additional components and levels of incorporation thereof will depend on the physical form of the composition, and the precise nature of the cleaning, disinfecting and/or whitening operation for which it is to be used. It will be understood that some of the adjunct additives noted below will have photoactive and/or electron acceptor properties, but it will be further understood that such additives will not replace the components noted above.

Suitable photocatalyzable consumer product compositions, and adjunct additives therefor, are described in detail in U.S. Application Ser. No. 61/930,993, filed Jan. 24, 2014, entitled “CONSUMER PRODUCT COMPOSITIONS” (Attorney Docket No. 13057P).

### Methods of Use

The present invention further relates to methods of using the systems of the present invention to provide benefits such as cleaning surfaces, bleaching stains (including whitening teeth) disinfecting and/or sanitizing surfaces, removing biofilm from surfaces, and the like.

As such, the present invention encompasses a method of treating a surface, the method comprising the steps of contacting the surface with a system of the present invention and exposing the surface/system to light, preferably having a wavelength greater than about 350 nm. The light utilized can be from a natural or artificial source.

The present invention further encompasses a method of bleaching a stain, the method comprising the steps of contacting the stain with a system of the present invention and exposing the system to light, preferably having a wavelength greater than about 350 nm.

The present invention further encompasses a method of disinfecting a surface, the method comprising the steps of contacting the surface with a system of the present invention and exposing the system to light, preferably having a wavelength greater than about 350 nm.

The present invention further encompasses a method of removing biofilm from a surface, the method comprising the steps of contacting the biofilm with a system of the present invention and exposing the system to light, preferably having a wavelength greater than about 350 nm.

The present invention also relates to a method for cleaning a stained fabric comprising contacting a stained fabric in need of cleaning with the system, described in detail above, having at least 0.001 ppm of a photoactivator, described in detail above, followed by exposing the surface of the treated fabric to a source of light having a minimal wavelength range of greater than about 300 nanometers, preferably greater than about 350 nanometers, preferably greater than about 400 nm, up to about 550 nanometers, preferably up to about 500 nanometers.

The present invention further relates to a method for cleaning a surface comprising contacting a surface in need of cleaning with the system, described in detail above, having at least 0.001 ppm of a photoactivator, described in detail above, followed by exposing the surface to a source of light having a minimal wavelength range of greater than about 300 nanometers, preferably greater than about 350 nanometers, up to about 550 nanometers, preferably up to about 500 nanometers.

The present invention further relates to a method for treating or cleaning oral cavity, including teeth or dentures (inside or outside the oral cavity), comprising contacting the oral cavity (including teeth or dentures) in need of treatment or cleaning with the system, described in detail above, having at least 0.001 ppm of a photoactivator, described in detail above, followed by exposing the teeth or dentures to a source of light having a minimal wavelength range of greater than about 300 nanometers, preferably greater than about 350 nanometers, up to about 550 nanometers, preferably up to about 500 nanometers.

### Packaging

The compositions of the system of the present invention may be packed in any suitable packaging for delivering the compositions for use. It will be understood, however, that the package may be structured to prevent the photoactivator from absorbing light and, therefore, activation before use. In one aspect, the package can be opaque. In another aspect, the package can be a transparent or translucent package made of glass or plastic so that consumers can see the photocatalyzable consumer product compositions throughout the packaging. In another aspect, the package may include one or more windows which may be opened to allow the consumer to see the composition and/or activate the composition prior to use and subsequently closed to prevent the photoactivator from absorbing light during storage. In one preferred aspect, the package may be comprised of polyethylene terephthalate, high-density polyethylene, low-density polyethylene, or combinations thereof. Furthermore, preferably, the package may be dosed through a cap at the top of the package such that the composition exits the bottle through an opening in the cap. In one aspect, the opening in the cap may also contain a screen to help facilitate dosing.

In another aspect, the package may comprise multiple compartments, preferably two compartments, with a first composition in a first compartment and a second composition in a second compartment. It will be understood that the photoactivator, electron acceptor and benefit active precursor may be included in either or both of the first and second compartments. In one preferred aspect, the first composition may comprise the photoactivator and the second composition may comprise the electron acceptor and benefit active precursor.

It should be understood that every maximum numerical limitation given throughout this specification would include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A system for treating a surface, the system comprising:
(a) a first composition comprising a water soluble organic photoactivator; and
(b) a second composition comprising:
(i) an electron acceptor which accepts an electron from the photoactivator when the photoactivator is in a photo-excited state and/or reduced state; and
(ii) a benefit active precursor which converts into a benefit active agent via electron transfer.

2. The system of claim 1, wherein the first composition is disposed in a first compartment and the second composition is disposed in a second compartment of a multi-compartment package.

3. The system of claim 1, wherein the electron acceptor is selected from the group consisting of organic species containing nitrogen, organic species containing sulfur, organic species containing oxygen, organic species containing phosphorus, anions of inorganic salts, and mixtures thereof.

4. The system of claim 1, wherein the electron acceptor is selected from the group consisting of viologens, 2,2′ bipyridinium, para-Benzoquinone, 2,3-Dichloro-5,6-dicyano-p-benzoquinone, Tetrahydroxy-1,4-quinone hydrate, 2,5-di-tert-butylhydroquinone, tert-Butylhydroquinone, Anthraquinone, Diaminoanthroquinone, Anthraquinone-2-sulfonic acid, Anthracene, Dicyanobenzene, Chloropentaamine cobalt dichloride, Silver nitrate, Iron Sulfate, Titanium Dioxide, Zinc Oxide, Cadmium Selenide, Thiamine hydrochloride, Thiamine pyrophosphate, Ammonium persulfate, Sodium persulfate, Potassium persulfate, (2,2,6,6-Tetramethylpiperidin-1-yl)oxy, Dimethylthiourea, Tetranitromethane, Lithium acetoacetate, Oxaloacetic acid, Sodium ascorbate, 2,6-Dicholorophenolindophenol, 4-methoxyphenol, 4-Methylmorpholine N-oxide, 4-tert-Butylcatechol, Allopurinol, Pyridoxal 5′-phosphate, pyridoxal hydrochloride, Sodium benzoate, Sodium Nitrate, Sodium Nitrite, Diatomic Oxygen, and mixtures thereof.

5. The system of claim 1, wherein the benefit active precursor has the formula:
A[XOₙ]ₘ
wherein
A is selected from the group consisting of monovalent cations, divalent cations, and trivalent cations;
X is selected from the group consisting of chlorine, bromine, iodine, and mixtures thereof;
n is 1, 2, 3, or 4; and
m is 1, 2, or 3.

6. The system of claim 5, wherein A is selected from the group consisting of Aluminum, Barium, Calcium, Cobalt, Chromium, Copper, Iron, Lithium, Potassium, Rubidium, Magnesium, Manganese, Molybdenum, Nickel, Sodium, Titanium, Vanadium, Zinc, ammonium, alkyl-ammonium, aryl-ammonium, and mixtures thereof.

7. The system of claim 6, wherein A is selected from the group consisting of lithium, sodium, potassium, magnesium, calcium, ammonium, and mixtures thereof.

8. The system of claim 5, wherein n is 2, 3, or 4.

9. The system of claim 1, wherein the photoactivator comprises less than about 35%, by weight of photoactivator, of a photoactive moiety.

10. The system of claim 1, wherein the photoactivator comprises less than about 2%, by weight of photoactivator, of a photoactive moiety.

11. The system of claim 1, wherein the photoactivator can be activated to the photo-excited state by excitation with incident radiation of a wavelength between about 350 nm and about 750 nm.

12. The system of claim 1, wherein the photoactivator can be activated to the photo-excited state by excitation with incident radiation of a wavelength between about 350 nm and about 420 nm.

13. The system of claim 1, wherein the photo-excited state of the photoactivator has an energy greater than about 100 kJ/mol more than a ground state of the photoactivator.

14. The system of claim 1, wherein the water soluble organic photoactivator comprises a photoactive moiety is selected from the group consisting of 1,1′-biphenyl-4,4′-diamine, 1,1′-biphenyl-4-amine, benzophenone, 1,1′-biphenyl-4,4′-diol, 1,1′-biphenyl-4-amine, 1,1′-biphenyl-4-ol, 1,1′:2′,1″-terphenyl, 1,1′:3′,1″-terphenyl, 1,1′:4′,1″:4″,1′″-quaterphenyl, 1,1′:4′,1″-terphenyl, 1,10-phenanthroline, 1,1′-biphenyl, 1,2,3,4-dibenzanthracene, 1,2-benzenedicarbonitrile, 1,3-isobenzofurandione, 1,4-naphthoquinone, 1,5-naphthalenediol, 10H-phenothiazine, 10H-phenoxazine, 10-methylacridone, 1-acetonaphthone, 1-chloroanthraquinone, 1-hydroxyanthraquinone, 1-naphthalenecarbonitrile, 1-naphthalenecarboxaldehyde, 1-naphthalenesulfonic acid, 1-naphthalenol, 2(1H)-quinolinone, 2,2′-biquinoline, 2,3-naphthalenediol, 2,6-dichlorobenzaldehyde, 21H,23H-porphine, 2-aminoanthraquinone, 2-benzoylthiophene, 2-chlorobenzaldehyde, 2-chlorothioxanthone, 2-ethylanthraquinone, 2H-1-benzopyran-2-one, 2-methoxythioxanthone, 2-methyl-1,4-naphthoquinone, 2-methyl-9(10-methyl)-acridinone, 2-methylanthraquinone, 2-methylbenzophenone, 2-naphthalenamine, 2-naphthalenecarboxylic acid, 2-naphthalenol, 2-nitro-9(10-methyl)-acridinone, 9(10-ethyl)-acridinone, 3,6-qcridinediamine, 3,9-dibromoperylene, 3,9-dicyanophenanthrene, 3-benzoylcoumarin, 3-methoxy-9-cyanophenanthrene, 3-methoxythioxanthone, 3′-methylacetophenone, 4,4′-dichlorobenzophenone, 4,4′-dimethoxybenzophenone, 4-bromobenzophenone, 4-chlorobenzophenone, 4′-fluoroacetophenone, 4-methoxybenzophenone, 4′-methylacetophenone, 4-methylbenzaldehyde, 4-methylbenzophenone, 4-phenylbenzophenone, 6-methylchromanone, 7-(diethylamino)coumarin, 7H-benz[de]anthracen-7-one, 7H-benzo[c]xanthen-7-one, 7H-furo[3,2-g][1]benzopyran-7-one, 9(10H)-acridinone, 9(10H)-anthracenone, 9(10-methyl)-acridinone, 9(10-phenyl)-acridinon, 9,10-anthracenedione, 9-acridinamine, 9-cyanophenanthrene, 9-fluorenone, 9H-carbazole, 9H-fluoren-2-amine, 9H-fluorene, 9H-thioxanthen-9-ol, 9H-thioxanthen-9-one, 9H-thioxanthene-2,9-diol, 9H-xanthen-9-one, acetophenone, acridene, acridine, acridone, anthracene, anthraquinone, anthrone, α-tetralone, benz[a]anthracene, benzaldehyde, benzamide, benzo[a]coronene, benzo[a]pyrene, benzo[f]quinoline, benzo[ghi]perylene, benzo[rst]pentaphene, benzophenone, benzoquinone, 2,3,5,6-tetramethyl, chrysene, coronene, dibenz[a,h]anthracene, dibenzo[b,def]chrysene, dibenzo[c,g]phenanthrene, dibenzo[def,mno]chrysene, dibenzo[def,p]chrysene, DL-tryptophan, fluoranthene, fluoren-9-one, fluorenone, isoquinoline, methoxycoumarin, methylacridone, michler's ketone, naphthacene, naphtho[1,2-g]chrysene, N-methylacridone, p-benzoquinone, p-benzoquinone, 2,3,5,6-tetrachloro, pentacene, phenanthrene, phenanthrenequinone, phenanthridine, phenanthro[3,4-c]phenanthrene, phenazine, phenothiazine, p-methoxyacetophenone, pyranthrene, pyrene, quinoline, quinoxaline, riboflavin 5′-(dihydrogen phosphate), thioxanthone, thymidine, xanthen-9-one, xanthone, and mixtures thereof.

15. The system of claim 1, wherein the water soluble organic photoactivator comprises a photoactive moiety selected from the group consisting of xanthone, xanthene, thioxanthone, thioxanthene, phenothiazine, fluorescein, benzophenone, alloxazine, isoalloxazine, flavin, and mixtures thereof.

16. The system of claim 15, wherein the photoactive moiety is thioxanthone.

17. The system of claim 1, wherein the water soluble organic photoactivator comprises a hydrophilic moiety selected from the group consisting of alkylene oxide oligimers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, polysaccharides, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphoinic acid, ethylene imine, and mixtures thereof.

18. The system of claim 1, wherein the benefit active precursor is an oxyhalite.

19. The system of claim 1, wherein the benefit active precursor is selected from the group consisting of chlorite salts, chlorate salts, bromite salts, bromate salts, iodite salts, iodate salts, and mixtures thereof.

20. The system of claim 1, wherein the benefit active precursor is selected from the group consisting of chlorite salts, chlorate salts, and mixtures thereof.

21. The system of claim 1, wherein the benefit active precursor is selected from the group consisting of sodium chlorite, sodium bromite sodium iodite, potassium chlorite, potassium bromite, potassium iodite, sodium chlorate, sodium bromate, sodium iodate, potassium chlorate, potassium bromate, potassium iodate, sodium hypochlorite, sodium hypobromite, sodium hypoiodite, sodium perchlorate, potassium perchlorate, and mixtures thereof.

22. The system of claim 1, wherein the benefit active precursor is sodium chlorite.

23. A method of treating a surface, the method comprising the steps of:
(a) applying to the surface a first composition comprising a water soluble organic photoactivator;
(b) applying to the surface a second composition comprising:
(i) an electron acceptor which accepts an electron from the photoactivator when the photoactivator is in a photo-excited state and/or reduced state; and
(ii) a benefit active precursor which converts into a benefit active agent via electron transfer; and
(c) exposing the surface to light.

24. The method of claim 18, wherein the light has a wavelength greater than about 350 nm, preferably between about 350 nm and about 750 nm, more preferably between about 350 nm and about 420 nm.

25. The method of claim 18, wherein the light has a wavelength between about 350 nm and about 750 nm.
